(19) United States
(12) Patent Application Publication
(10) Pub. No.: US 2011/0022120 A1
(43) Pub. Date: Jan. 27, 2011

(54) Magnetic Attachment Arrangement for Implantable Device

(75) Inventors: Geoffrey R. Ball, Axams (AT); Peter Lampacher, Innsbruck (AT); Bernhard Jamnig, Innsbruck (AT); Martin Zimmerling, Patsch (AT); Gunrher Weidenholzer, Ottensheim (AT); Markus Nagl, Volders (AT); Wolfgang Amrhein, Ottensheim (AT)

Correspondence Address: Sunstein Kann Murphy & Timbers LLP, 125 SUMMER STREET, BOSTON, MA, 02110-1618, US

(73) Assignee: VIBRANT MED-EL HEARING TECHNOLOGY GMBH, Innsbruck (AT)

(21) Appl. No.: 12/839,887

(22) Filed: Jul. 20, 2010

Related U.S. Application Data
(60) Provisional application No. 61/227,632, filed on Jul. 22, 2009.

(51) Int. Cl. A61N 1/05

(52) U.S. Cl. 607/57

(57) ABSTRACT

A magnet arrangement is described for use in implantable devices. An implantable housing contains a portion of an implantable electronic system. A cylindrical implant magnet arrangement within the housing includes multiple adjacent magnetic sections wherein at least two of the magnetic sections have opposing magnetic orientations in opposite magnetic directions. There may also be a similar external housing having a corresponding magnet arrangement.

This application claims priority from U.S. Provisional Patent Application 61/227,632, filed Jul. 22, 2009, which is incorporated herein by reference.

### FIELD OF THE INVENTION

The present invention relates to medical implants, and more specifically to a permanent magnet arrangement for use in such implants.

### BACKGROUND ART

Some hearing implants such as Middle Ear Implants (MEI's) and Cochlear Implants (CI's) employ attachment magnets in the implantable part and an external part to hold the external part magnetically in place over the implant. For example, as shown in FIG. 1, a typical cochlear implant system may include an external transmitter housing 101 containing transmitting coils 102 and an external magnet 103. The external magnet 103 has a conventional coin-shape and a north-south magnetic dipole that is perpendicular to the skin of the patient to produce external magnetic field lines 104 as shown. Implanted under the patient's skin is a corresponding receiver assembly 105 having similar receiving coils 106 and an implanted internal magnet 107. The internal magnet 107 also has a coin-shape and a north-south magnetic dipole that is perpendicular to the skin of the patient to produce internal magnetic field lines 108 as shown. The internal receiver housing 105 is surgically implanted and fixed in place within the patient's body. The external transmitter housing 101 is placed in proper position over the skin covering the internal receiver assembly 105 and held in place by interaction between the internal magnetic field lines 108 and the external magnetic field lines 104. Rf signals from the transmitter coils 102 couple data and/or power to the receiving coil 106 which is in communication with an implanted processor module (not shown).

One problem arises when the patient undergoes Magnetic Resonance Imaging (MRI) examination. Interactions occur between the implant magnet and the applied external magnetic field for the MRI. As shown in FIG. 2, the direction magnetization [symbol] of the implant magnet 202 is essentially perpendicular to the skin of the patient. Thus, the external magnetic field [symbol] from the MRI may create a torque [symbol] the internal magnet 202, which may displace the internal magnet 202 or the whole implant housing 201 out of proper position. Among other things, this may damage the adjacent tissue in the patient. In addition, the external magnetic field [symbol] from the MRI may reduce or remove the magnetization [symbol] of the implant magnet 202 so that it may no longer be strong enough to hold the external transmitter housing in proper position. The implant magnet 202 may also cause imaging artifacts in the MRI image, there may be induced voltages in the receiving coil, and hearing artifacts due to the interaction of the external magnetic field [symbol] of the MRI with the implanted device. This is especially an issue with MRI field strengths exceeding 1.5 Tesla.

Thus, for existing implant systems with magnet arrangements, it is common to either not permit MRI or at most limit use of MRI to lower field strengths. Other existing solutions include use of a surgically removable magnets, spherical implant magnets (e.g. U.S. Pat. No. 7,566,296), and various ring magnet designs (e.g., U.S. Provisional Patent 61/227,632, filed Jul. 22, 2009). Among those solutions that do not require surgery to remove the magnet, the spherical magnet design may be the most convenient and safest option for MRI removal even at very high field strengths. But the spherical magnet arrangement requires a relatively large magnet much larger than the thickness of the other components of the implant, thereby increasing the volume occupied by the implant. This in turn can create its own problems. For example, some systems, such as cochlear implants, are implanted between the skin and underlying bone. The “spherical bump” of the magnet housing therefore requires preparing a recess into the underlying bone. This is an additional step during implantation in such applications which can be very challenging or even impossible in case of very young children.

Various complicated arrangements of magnetic elements have been described for use in therapeutic applications; see for example, U.S. Pat. No. 4,549,532 and U.S. Pat. No. 7,608,035. However, there is no suggestion that such therapeutic arrangements might potentially have any utility for magnetic attachment applications such as those described above.

### SUMMARY OF THE INVENTION

Embodiments of the present invention are directed to a magnet arrangement for use in implantable devices. An implantable housing contains a portion of an implantable electronic system. A cylindrical implant magnet arrangement within the housing includes multiple adjacent magnetic sections wherein at least two of the magnetic sections have opposing magnetic orientations in opposite magnetic directions.

For example, the magnetic sections may include an inner center disc having an inner magnetic orientation in an inner magnetic direction, and an outer radial ring having an outer magnetic orientation in an outer magnetic direction opposite to the inner magnetic direction. Or the magnetic sections may include multiple wedge sections formed together into a cylinder shape wherein adjacent wedge sections have opposing magnetic orientations in opposite magnetic directions. Or the magnet arrangement may include an inner center disc surrounded by outer radial ring having a plurality of wedge sections wherein adjacent wedge sections have opposing magnetic orientations in opposite magnetic directions.

Many embodiments also have an implant signal coil within the housing surrounding the implant magnet arrangement for receiving an implant communication signal. In some embodiments, there may be multiple implant magnet arrangements. There may also be a similar external housing having a corresponding magnet arrangement. The implantable electronic system may be, for example, a cochlear implant system, a middle ear implant system, or a bone conduction hearing implant system.

### BRIEF DESCRIPTION OF THE DRAWINGS

FIG. 1 shows a portion of a typical idealized cochlear implant which may be used in embodiments of the present invention.

FIG. 2 shows effects of an external magnetic field on an implanted portion of an implanted device which may be used in embodiments of the present invention.

FIG. 3 A-B shows an implant magnet arrangement according to embodiments of the present invention.

FIG. 4 shows how an embodiment of an implant magnet arrangement cooperates with a typical external device.

FIG. 5 shows how an embodiment of an implant magnet arrangement cooperates with another corresponding external magnet arrangement.

FIG. 6 shows an embodiment of an implant magnet having magnetically alternating pie-shaped magnetic sections.

FIG. 7 shows another embodiment similar to the one in FIG. 6 with an inner center disk.

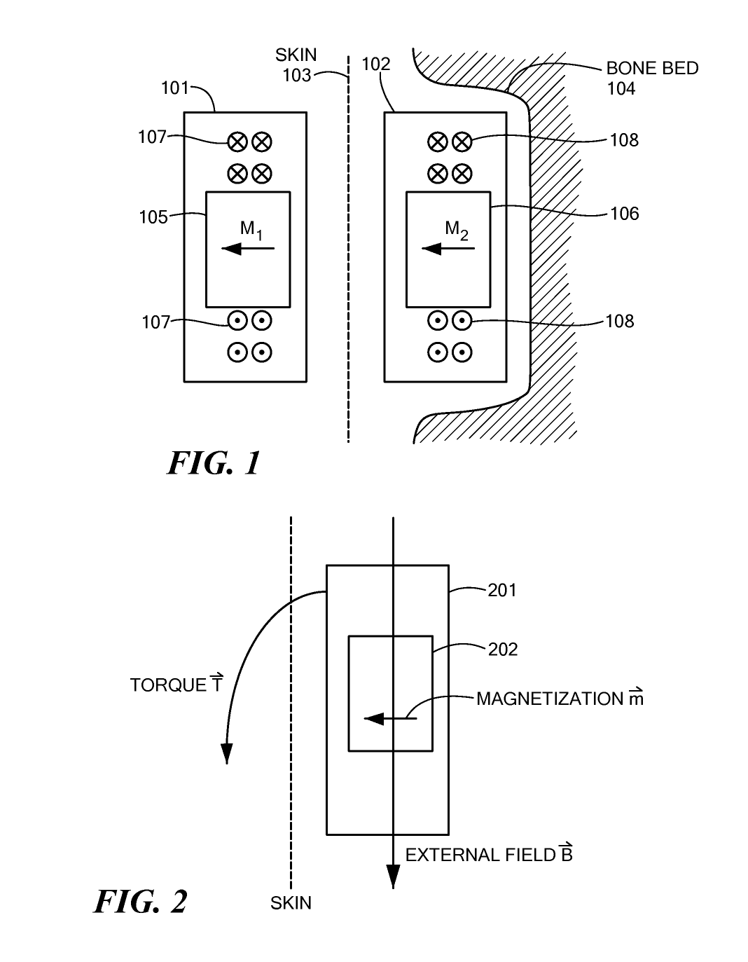

### DETAILED DESCRIPTION OF SPECIFIC EMBODIMENTS

Various embodiments of the present invention are directed to an improved magnet arrangement for implantable devices in the form of a cylindrical magnet having multiple adjacent magnetic sections wherein at least two of the magnetic sections have opposing magnetic orientations in opposite magnetic directions.

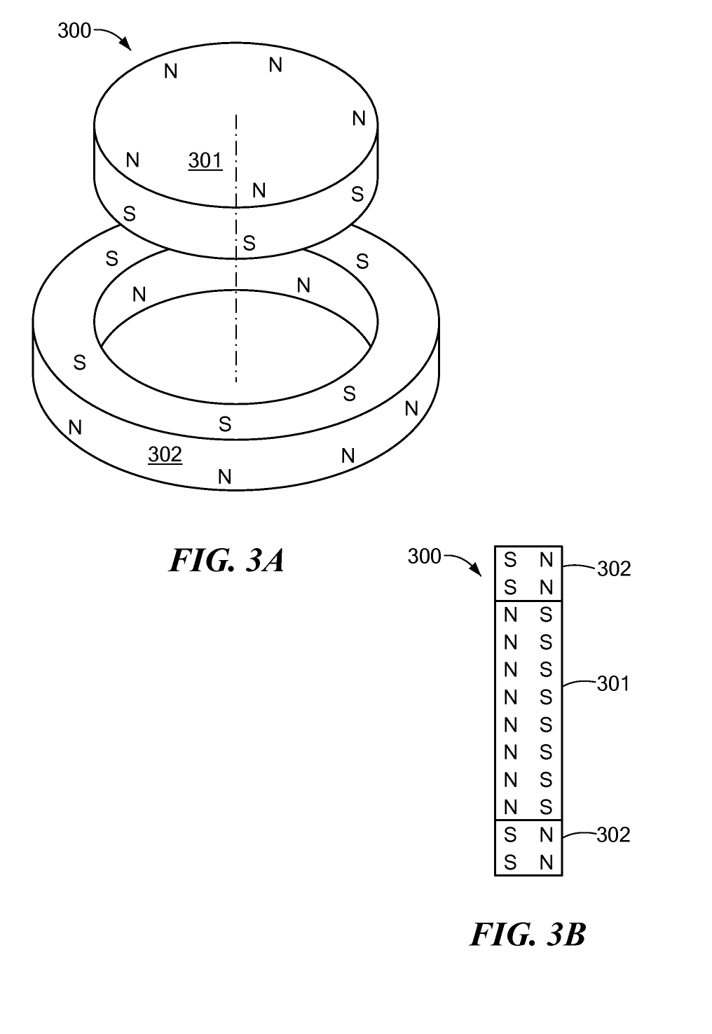

FIG. 3 A shows an exploded elevated view and FIG. 3 B shows a side view of an implant magnet arrangement 300 according to embodiments of the present invention. An implantable housing (e.g., implant housing 102) contains a portion of an implantable electronic system. The implantable electronic system may be, for example, a cochlear implant system, a middle ear implant system, or a bone conduction hearing implant system. A cylindrical implant magnet arrangement 300 within the housing includes an inner center disc section 301 having an inner magnetic orientation in an inner magnetic direction, and an outer radial ring section 302 having an outer magnetic orientation in an outer magnetic direction opposite to the inner magnetic direction.

With such an arrangement, the net magnetic field of the implant magnet arrangement 300 is much less than in the conventional cylindrical magnet of the prior art, while locally the magnetic fields are still effectively strong near the inner center disc section 301 and the outer radial ring section 302 so that there is no overall loss in the retention force of the implant magnet arrangement 300. Such a reduced net magnetic field of the implant magnet arrangement 300 also avoids the prior problems of the net magnetic fields adversely interacting with the implant signal coil and its communications signal and reduces the torque and imaging problems of the prior art with regards to MRI procedures. Moreover, the greater specificity of the magnetic structures of the implant magnet arrangement 300 compared with a simple disk magnet also provides improved centering capability with regards to the external component housing.

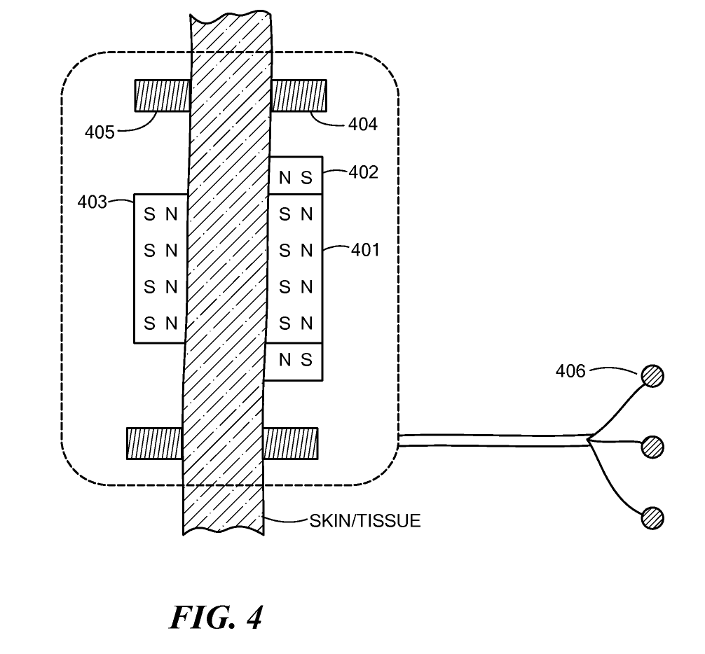

FIG. 4 shows how an embodiment of an implant magnet arrangement cooperates with a typical external device. A conventional cylindrical external magnet 403 interacts with an implant magnet having an inner center disc section 401 and an outer radial ring section 402 according to an embodiment of the invention. In this case, the external magnet 403 is similar in diameter to the inner center disc section 401 of the implant magnet so that their respective magnetic fields interact to provide the desired retention force to hold the external device in proper operating position. This allows external signal coil 405 to couple an implant communications signal containing data and power through to a corresponding implant coil 404. The implant communications signal received by the implant coil 404 then is coupled to other elements 406 of the implant system such as an implant processor of a cochlear implant, bone conduction transducer, or middle ear transducer. In some embodiments, there may be multiple implant magnet arrangements and corresponding external magnets.

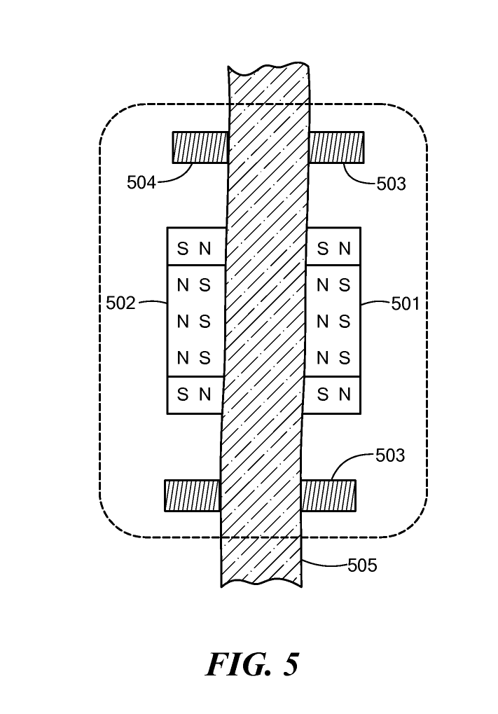

FIG. 5 shows how an embodiment of an implant magnet arrangement cooperates with another corresponding external magnet arrangement. In this case, the external magnet 502 also has inner and outer sections that correspond to similar sections of the implant magnet 501 to cooperate to hold the external device in proper operating position. In some embodiments, there may be multiple implant magnet arrangements. This allows an external signal coil 504 to couple an implant communications signal containing data and power across the skin 505 to a corresponding implant coil 503 for use by other elements of the implant system.

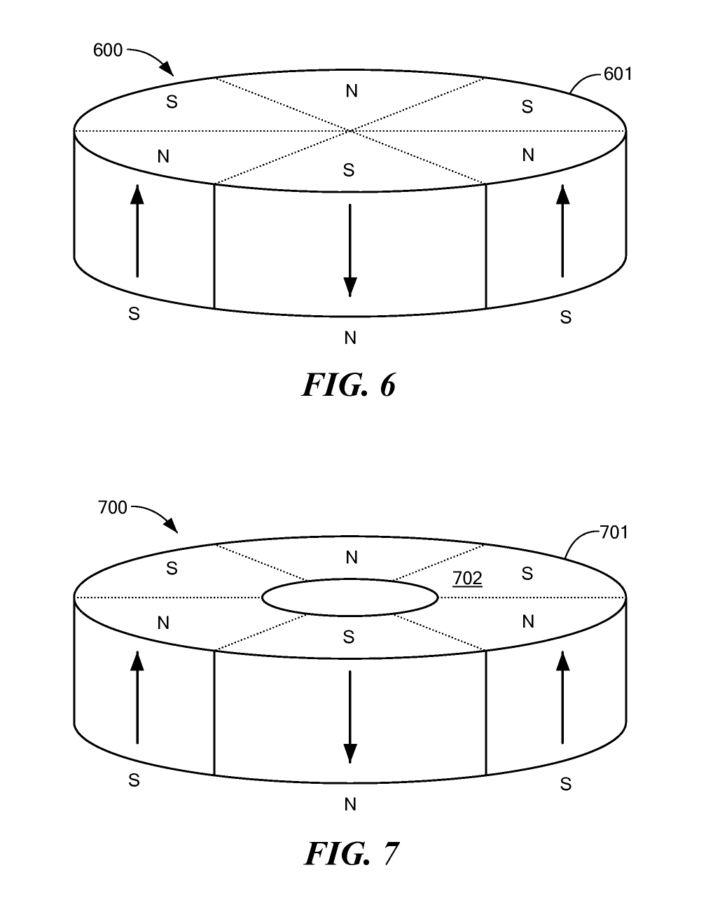

FIG. 6 shows another embodiment of the present invention where an implant magnet arrangement 600 includes axial magnetized wedge sections 601 with adjacent wedge sections having diametrically opposed magnetic orientation. The implant magnet arrangement 600 in FIG. 6 shows six magnetized wedge sections 601, but other embodiments may have different numbers of wedge sections so long as the overall net magnetic field of the arrangement as a whole is minimized. In addition, FIG. 7 shows another embodiment with an inner center disk 701 which may or may not be magnetized, surrounded by an outer radial ring 702 which is sub-divided into magnetized partial wedge sections 703 where adjacent wedge sections are oppositely magnetized. In such arrangements (or indeed, many of the above embodiments), between the individual magnetized sections there also may be narrow unmagnetized transition elements.

Embodiments such as the one shown in FIGS. 6 and 7 allow the external housing to be attached on the skin at a fixed specified angle, which can be useful for ensuring proper alignment of directional microphones. Of course, for some applications this might be seen as a drawback in that the external housing can be only be fixed at a limited number of specific angles depending on the numbers of wedge sections. For example, if the implant magnet arrangement 600 has four axial magnetized wedge sections 601, then the external part can only be rotated at an angle of 180°. With six magnetized wedge sections 601, the rotation angle is 120°.

Embodiments of the present invention such as those described above can be easily and directly implemented in existing products with corresponding size and geometry replacement magnets, either for the implanted magnet and/or the external magnet. Embodiments may usefully contain permanent magnetic material and/or ferro-magnetic material as well as other structural materials. These include without limitation magnetic ferrite materials such as Fe₃O₄, BaFe₁₂O₁₉ etc., compound materials such as plastic bonded permanent magnetic powder, and/or sintered material such as sintered NdFeB, SmCo, etc. Selection of the proper materials and arrangements may help avoid or reduce undesired eddy currents.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable device comprising:
an implant housing containing a portion of an implantable electronic system; and
a cylindrical implant magnet arrangement within the housing including a plurality of adjacent magnetic sections wherein at least two of the magnetic sections have opposing magnetic orientations in opposite magnetic directions.

2. An implantable device according to claim 1 wherein the magnetic sections include:
i. an inner center disc having an inner magnetic orientation in an inner magnetic direction, and
ii. an outer radial ring having an outer magnetic orientation in an outer magnetic direction opposite to the inner magnetic direction.

3. An implantable device according to claim 1 wherein the magnetic sections include a plurality of wedge sections formed together into a cylinder shape wherein adjacent wedge sections have opposing magnetic orientations in opposite magnetic directions.

4. An implantable device according to claim 1 wherein the magnet arrangement includes an inner center disc surrounded by outer radial ring having a plurality of wedge sections wherein adjacent wedge sections have opposing magnetic orientations in opposite magnetic directions.

5. An implantable device according to claim 1, further comprising:
an implant signal coil within the housing, surrounding the implant magnet arrangement for receiving an implant communication signal.

6. An implantable device according to claim 1, wherein the implantable housing contains a plurality of implant magnet arrangements.

7. An implantable device according to claim 1, wherein the implantable electronic system includes a cochlear implant system.

8. An implantable device according to claim 1, wherein the implantable electronic system includes a middle ear implant system.

9. An implantable device according to claim 1, wherein the implantable electronic system includes a bone conduction hearing implant system.

10. An external device for use with an implant system, the device comprising:
an external device housing containing external elements of an implantable electronic system; and
a cylindrical external magnet arrangement within the housing including a plurality of adjacent magnetic sections wherein at least two of the magnetic sections have opposing magnetic orientations in opposite magnetic directions.

11. An implantable device according to claim 10, wherein the magnetic sections include:
i. an inner center disc having an inner magnetic orientation in an inner magnetic direction, and
ii. an outer radial ring having an outer magnetic orientation in an outer magnetic direction opposite to the inner magnetic direction.

12. An implantable device according to claim 10, wherein the magnetic sections include a plurality of wedge sections formed together into a cylinder shape wherein adjacent wedge sections have opposing magnetic orientations in opposite magnetic directions.

13. An implantable device according to claim 10, wherein the magnet arrangement includes an inner center disc surrounded by an outer radial ring having a plurality of wedge sections wherein adjacent wedge sections have opposing magnetic orientations in opposite magnetic directions.

14. An external device according to claim 10, further comprising:
an external signal coil within the housing, surrounding the external magnet arrangement for transmitting an implant communication signal.

15. An external device according to claim 10, wherein the external device housing contains a plurality of external magnet arrangements.

16. An external device according to claim 10, wherein the implantable electronic system includes a cochlear implant system.

17. An external device according to claim 10, wherein the implantable electronic system includes a middle ear implant system.

18. An external device according to claim 10, wherein the implantable electronic system includes a bone conduction hearing implant system.
